(19) United States
(12) Patent Application Publication
(10) Pub. No.: US 2026/0105617 A1
(43) Pub. Date: Apr. 16, 2026

(54) INFORMATION PROCESSING APPARATUS, RADIATION IMAGING SYSTEM, INFORMATION PROCESSING METHOD, AND NON-TRANSITORY COMPUTER-READABLE STORAGE MEDIUM

(71) Applicant: CANON KABUSHIKI KAISHA, Tokyo (JP)

(72) Inventor: TAKUYA TSUKADA, Chiba (JP)

(21) Appl. No.: 19/351,538

(22) Filed: Oct. 7, 2025

(30) Foreign Application Priority Data
Oct. 15, 2024 (JP) 2024-180340

(51) Int. Cl. G06T 7/246; A61B 6/46; G06T 7/00

(52) U.S. Cl. CPC G06T 7/251; A61B 6/463; A61B 6/467; G06T 7/0014; G06T 2207/20081

(57) ABSTRACT

An information processing apparatus, which processes a radiation image obtained via radiation imaging of a subject, includes: an analysis unit configured to perform an analysis of movement of the subject in an optical image obtained from a first optical imaging apparatus; a determination unit configured to perform a determination of whether to display the optical image on a display unit, based on the analysis; and a display control unit configured to perform control display of the display unit, based on the determination.

### BACKGROUND

### Field of the Technology

The present disclosure relates to an information processing apparatus, a radiation imaging system, an information processing method, and a non-transitory computer-readable storage medium.

### Description of the Related Art

As an imaging apparatus used in medical imaging diagnostics and non-destructive examination using radiation, a radiation imaging apparatus that uses a flat panel detector (FPD) made of a semiconductor material has become commonly used. In medical imaging diagnostics, for example, such a radiation imaging apparatus is used as a digital imaging apparatus for still image capturing such as general radiography and moving image capturing such as fluoroscopy.

In recent years, in radiation imaging, the imaging location is imaged with an optical camera and imaging assistance using the optical camera is performed. For example, in Japanese Patent Laid-Open No. 2012-24399, a technology is described for performing radiation imaging while checking the imaging status using an optical image.

In a radiation imaging system such as that described in Japanese Patent Laid-Open No. 2012-24399, an optical image is displayed on a display, but at timings when it is not necessary to check the imaging status such as when checking the captured radiation image, there are times when the optical image becomes unnecessary and is not displayed. Also, if a subject is shown in the optical image, there is an increased possibility of it being seen by people other than the examiner. Thus, taking into account the privacy of the subject, there is a demand for the optical image to be displayed as seldom as possible.

However, when the optical image is not displayed, monitoring of the subject cannot be performed via the optical image. Thus, there is a possibility of being delayed in noticing a change in the condition of the subject or noticing an emergency.

### SUMMARY

The present disclosure has been made in light of the problems described above and enables realization of information processing technology for an efficient imaging flow that can detect a change in the condition of a detected subject by displaying an optical image in a case where a change in the condition of the subject, such as movement exceeding a predetermined movement amount, even in a situation in which the optical image is not displayed.

According to one aspect of the present disclosure, there is provided an information processing apparatus that processes a radiation image obtained via radiation imaging of a subject, comprising: an analysis unit configured to perform an analysis of movement of the subject in an optical image obtained from a first optical imaging apparatus; a determination unit configured to perform a determination of whether to display the optical image on a display unit, based on the analysis; and a display control unit configured to perform control display of the display unit, based on the determination.

Further features of the present disclosure will become apparent from the following description of exemplary embodiments with reference to the attached drawings.

### BRIEF DESCRIPTION OF THE DRAWINGS

FIG. 1 is a diagram illustrating an example of the basic configuration of a radiation imaging system according to a first embodiment.

FIG. 2 is a diagram illustrating a detailed configuration of an optical image analysis unit 105 according to the first embodiment.

FIG. 3 is a flowchart illustrating display determination processing for an optical image using an optical imaging apparatus 140 according to a first embodiment.

FIG. 4 is a flowchart illustrating analysis processing for an optical image obtained from the optical imaging apparatus 140 according to the first embodiment.

FIG. 5 is a diagram illustrating an example of configuration of a radiation imaging system according to a second embodiment.

FIG. 6 is a flowchart illustrating display determination processing for an optical image using a monitoring camera 170 according to the first embodiment.

FIG. 7 is a flowchart illustrating analysis processing for sensor data obtained from the monitoring camera 170 according to the first embodiment.

### DESCRIPTION OF THE EMBODIMENTS

Hereinafter, embodiments will be described in detail with reference to the attached drawings. Note, the following embodiments are not intended to limit the scope of the claims. Multiple features are described in the embodiments, but it is not the case that all such features are required, and multiple such features may be combined as appropriate. Furthermore, in the attached drawings, the same reference numerals are given to the same or similar configurations, and redundant description thereof is omitted.

### First Embodiment

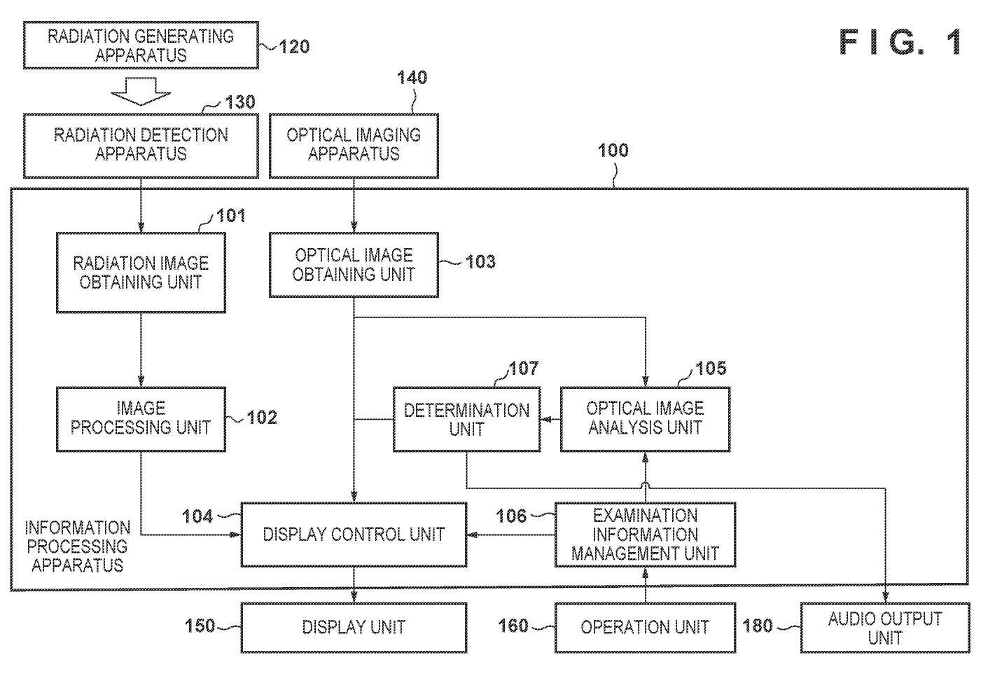

FIG. 1 is a diagram illustrating an example of the basic configuration of a radiation imaging system according to the present embodiment. The radiation imaging system includes an information processing apparatus 100, a radiation generating apparatus 120, a radiation detection apparatus 130, an optical imaging apparatus 140, a display unit 150, and an operation unit 160. The information processing apparatus 100 controls radiation imaging using the radiation detection apparatus 130 and the radiation generating apparatus 120. The information processing apparatus 100 is connected in a communication-enabling manner to the radiation detection apparatus 130, the radiation generating apparatus 120, and the optical imaging apparatus 140.

The radiation generating apparatus 120 is provided with a radiation tube for generating radiation and emits radiation at a subject such as a patient. The radiation detection apparatus 130 generates image data (radiation image) based on the radiation emitted from the radiation generating apparatus 120. The radiation detection apparatus 130, for example, may be constituted by a flat panel detector (FPD) or the like. The radiation detection apparatus 130 includes a sensor panel including a plurality of pixels arranged to form a plurality of rows and a plurality of columns. The plurality of pixels of the sensor panel function as a detection element for detecting radiation. The radiation detection apparatus 130 detects radiation that is emitted from the radiation generating apparatus 120 and that passes through the subject (not illustrated) and generates and outputs image data corresponding to the detected radiation. The image data generated by the radiation detection apparatus 130 may also be referred to as a medical image or a radiation image.

Specifically, the radiation detection apparatus 130 detects the radiation that passes through the subject as a charge corresponding to the transmitted radiation amount. For example, the radiation detection apparatus 130 may use a direct conversion sensor that directly converts radiation into a charge using a-Se or the like to convert radiation into a charge or an indirect conversion sensor using a scintillator such as CsI and a photoelectric conversion element such as a-Si to convert radiation into visible light. Also, the radiation detection apparatus 130 A/D converts the detected charge to generate image data and outputs the image data to the information processing apparatus 100.

### Information Processing Apparatus 100

The information processing apparatus 100 processes the radiation image obtained via radiation imaging of the subject. The information processing apparatus 100 is connected to the radiation generating apparatus 120, the radiation detection apparatus 130, and the optical imaging apparatus 140 via a wired or wireless network or dedicated line. The information processing apparatus 100 may be configured of a computer including a processor, memory, and the like and may be configured as a dedicated control apparatus for the radiation imaging system. The processor of the information processing apparatus 100 may be a central processing unit (CPU). Also, the processor may be, for example, a micro processing unit (MPU), a graphical processing unit (GPU), a field-programmable gate array (FPGA), or the like.

Each function of the information processing apparatus 100 may be implemented by the processor such as a CPU or a MPU executing a software module stored in an internal storage unit of the information processing apparatus 100. Note that the processor may be, for example, a GPU, an FPGA, or the like. Also, each function of the information processing apparatus 100 may be configuration of a circuit or the like that performs a specific function such as an ASIC or the like. For example, each functional configuration of the information processing apparatus 100 may be implemented by dedicated hardware such as an ASIC or the like.

The operation unit 160 includes, for example, a keyboard, a pointing device (for example, a mouse), a touch panel, or a similar input apparatus and receives various types of input from an operator. The information processing apparatus 100 sets the imaging condition for the radiation on the basis of the examination information received via the operation unit 160 and controls the timing for the radiation generating apparatus 120 to generate radiation on the basis of the set imaging condition.

### Radiation Image Obtaining Unit 101

The radiation image obtaining unit 101 controls the timing at which the radiation detection apparatus 130 captures image data and the output timing and receives the image data generated by the radiation detection apparatus 130.

### Image Processing Unit 102

The image processing unit 102 executes image processing on the image data received by the radiation image obtaining unit 101. The image processing unit 102 applies image processing for diagnosis such as tone processing, emphasis processing, noise reduction processing, and the like on the image data and performs image generation in accordance with the diagnosis.

### Display Control Unit 104

The display unit 150 is implemented by a liquid crystal display, a cathode ray tube (CRT) display, or the like, for example. The display unit 150 displays various types of information under the control of the display control unit 104. The display control unit 104 can performs display control of the display of the display unit 150 and can cause the display unit 150 to display radiation images of before and after image processing by the image processing unit 102, subject information, and the like. The display control unit 104 performs display control to cause the display unit 150 to display the post-image-processing image processed by the image processing unit 102.

The display control unit 104 provides a graphical user interface (GUI) using the display unit 150 and receives instructions from the operator via the operation unit 160.

### Examination Information Management Unit 106

The examination information management unit 106 manages a plurality of pieces of examination information from a plurality of radiation imagings. The examination information management unit 106 causes the display unit 150 to display the plurality of pieces of examination information in a list format via the display control unit 104. The examination information management unit 106 causes the operator to select one piece of examination information from among the plurality of pieces of examination information displayed in a list format on the basis of an input from the operation unit 160 and sets the selected examination information as the examination information to be used in the radiation imaging. The examination information management unit 106 outputs the set examination information to an optical image analysis unit 105 and the display control unit 104. Note that the operator may directly input the examination information from the operation unit 160 to the output of the optical image analysis unit 105 and the display control unit 104. The information processing apparatus 100 can control the timing of when a radiation image or an optical image is obtained on the basis of the set examination information. For example, the information processing apparatus 100 sets the imaging condition and the like for controlling the radiation imaging on the basis of the examination information received via the operation unit 160 and controls the timing at which the radiation generating apparatus 120 generates radiation and the generation of image data (radiation image) by the radiation detection apparatus 130. The radiation detection apparatus 130 detects the radiation generated by the radiation generating apparatus 120 and outputs image data (radiation image) corresponding to the detected radiation to the information processing apparatus 100.

Here, the examination information managed by the examination information management unit 106 may include the department name of the source of the request for examination, the examination ID, the examination item, the imaging conditions for radiation imaging (imaging protocol), and patient information (subject information) relating to the subject. Also, the examination information may include information for identifying the imaging environment (for example, a standing stand, a decubitus table, the radiation detection apparatus 130 (FPD) itself, or the like), the imaging site (for example, the front of the chest, the side of the abdomen, or the like), and the imaging orientation (for example, standing, decubitus, or the like).

### Optical Imaging Apparatus 140

The radiation imaging system is provided with the optical imaging apparatus 140 (for example, an optical camera) that captures an optical image of a subject. The optical imaging apparatus 140 performs video capture as the optical image of the subject. The optical imaging apparatus 140, for example, may be disposed at or near the radiation generating apparatus 120 so that the imaging site of the subject for radiation imaging can be imaged. Also, the optical imaging apparatus 140 is not limited to a single optical imaging apparatus 140 and a plurality of optical imaging apparatuses 140 may be used. For example, the optical imaging apparatuses 140 may be disposed in different directions for imaging the subject so that an optical image from the front direction and the side direction can be obtained.

The information processing apparatus 100 controls the conditions, timing, frame rate, and the like for the optical imaging apparatus 140 to obtain an optical image.

### Optical Image Obtaining Unit 103

The optical image obtaining unit 103 obtains the optical image from the optical imaging apparatus 140. In a case where the plurality of optical imaging apparatuses 140 are provided, the optical image obtaining unit 103 obtains optical images from the plurality of optical imaging apparatuses 140.

The optical image obtaining unit 103 outputs the optical image obtained from the optical imaging apparatus 140 to the display control unit 104.

The display control unit 104 performs display control to cause the display unit 150 to display the optical image output from the optical image obtaining unit 103. In a case where a plurality of optical images are output, the display control unit 104 performs display control to cause the display unit 150 to display the plurality of optical images. The display control unit 104 attaches information (additional information) to be display other than the optical image and controls the content to be displayed using the display unit 150. The additional information may include the imaging conditions (imaging protocol) for radiation imaging. When an optical image is displayed, the display control unit 104 may also cause the display unit 150 to display additional information to notify of subject movement. The additional information to be displayed on the display unit 150 may include one of a message for notifying of subject movement, an icon (mark or symbol for notification), or a combination of a message and an icon. In a case where the movement of the subject exceeds a predetermined movement amount, the display control unit 104 may cause the display unit 150 to change the display color of the additional information and display it. Here, the predetermined movement amount is a threshold that can be changed via the settings. The optical image analysis unit 105 described below determines that the subject is a moving body when the movement of the subject detected by moving body detection processing exceeds the threshold.

Note that the additional information is not limited to information to be displayed on the display unit 150 and may be sound output from an audio output unit 180 constituted by a speaker or the like.

In a case where the display control unit 104 displays an optical image, a determination unit 107 may output a notification sound for notifying of subject movement from the audio output unit 180. Also, in a case where the subject movement exceeds the predetermined movement amount and increases, the determination unit 107 may change the type of the notification sound or increase the volume of the notification sound and output it from the audio output unit 180. Also, changing the type of the notification sound and changing the volume of the notification sound may be performed in combination.

### Optical Image Analysis Unit 105

The optical image analysis unit 105 analyzes the movement of the subject in the optical image obtained from the optical imaging apparatus 140 (first optical imaging apparatus). In a case where a plurality of different human bodies are detected in a frame of the optical image via the image processing described below or human body detection processing based on inference processing using a learned model, the optical image analysis unit 105 may obtain feature information of the plurality of detected human bodies and detect one of the plurality of human bodies as the subject on the basis of the obtained feature information.

An example of a case in which a plurality of different human bodies are detected in a frame includes a case in which the operator and the subject have both entered the frame of the optical image, for example. In such a case, the feature information of the operator, for example, may be known operator feature information such as a white coat or hospital-specific clothes type, color, name card, and the like. Also, the features of the subject may be known subject feature information such as subject information (for example, adult, child, gender, height, or other physical trait), examination clothes worn by subjects, and the like obtained from the examination information.

In a case where a plurality of different human bodies are detected in a frame of the optical image via human body detection processing, the optical image analysis unit 105 obtains feature information of the plurality of human bodies and detects one of the plurality of human bodies as the subject on the basis of the obtained feature information. For example, in a case where the feature information of both the operator and the subject are obtained or the feature information of only the subject is obtained, the optical image analysis unit 105 detects the subject from the plurality of human bodies on the basis of the obtained feature information of the subject.

Alternatively, in a case where the feature information of only the operator is obtained, the optical image analysis unit 105 detects the human body left after excluding the operator from the plurality of human bodies as the subject on the basis of the obtained feature information of the operator.

Also, the optical image analysis unit 105 uses the optical image obtained from the optical image obtaining unit 103 and the examination information obtained from the examination information management unit 106 to analyze the subject position (positioning position) in the imaging environment, the subject imaging site, and the imaging orientation and determines whether they match the setting content of the examination information. Here, the examination information includes information for identifying the imaging environment (for example, a standing stand, a decubitus table, the radiation detection apparatus 130 (FPD) itself, or the like), the imaging site (for example, the front of the chest, the side of the abdomen, or the like), and the imaging orientation (for example, standing, decubitus, or the like). The optical image analysis unit 105 compares the analysis information obtained from the analysis of the optical image and various pieces of information included in the examination information and determines whether or not they match the setting content of the examination information.

The optical image analysis unit 105 determines whether the subject exists in a predetermined position in the imaging environment set in the examination information via the image processing described below or human body detection processing based on inference processing using a learned model. Also, the optical image analysis unit 105 determines whether or not the imaging site and imaging orientation of the subject obtained by analysis of the optical image match the imaging site and imaging orientation of the subject set in the examination information.

### Determination Unit 107

The determination unit 107 determines whether to display the optical image on the display unit 150 on the basis of the analysis result of the optical image analysis unit 105 or the analysis result of a sensor data analysis unit 109 described below in the second embodiment. The display control unit 104 controls the display of the display content of the display unit 150 on the basis of the determination result of the determination unit 107.

### Detailed Configuration of Optical Image Analysis Unit 105

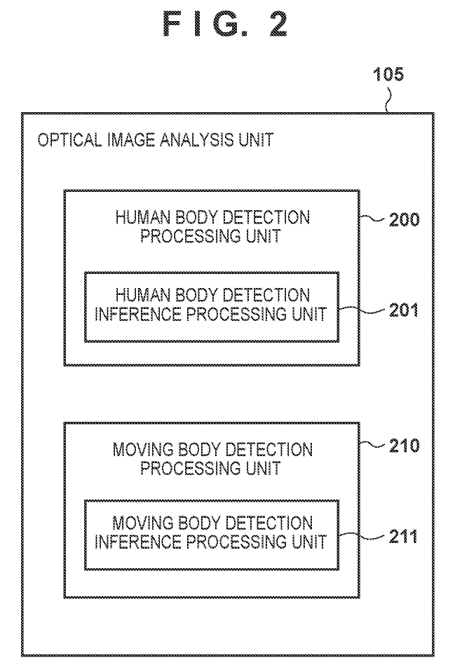

FIG. 2 is a diagram illustrating a detailed configuration of the optical image analysis unit 105 according to the present embodiment. The optical image analysis unit 105 includes a human body detection processing unit 200, a human body detection inference processing unit 201, a moving body detection processing unit 210, and a moving body detection inference processing unit 211. The human body detection processing unit 200 includes the human body detection inference processing unit 201, and the moving body detection processing unit 210 includes the moving body detection inference processing unit 211.

The human body detection processing unit 200 executes human body detection processing to determine whether a subject (human body) is captured in a frame of the optical image obtained from the optical image obtaining unit 103. Hereinafter, subject detection may be referred to as human body detection.

The human body detection processing unit 200, for example, may determine whether a subject is captured in an optical image via image processing of the optical image. The human body detection processing unit 200 may, for example, as image processing of the optical image, extract the contour of a human body via contour extraction or may identify a candidate region of a human body from the feature amount of each region obtained by dividing the image (image frame) into a plurality of regions. Also, pattern matching using human body comparison patterns may be performed to detect a human body shown in the image (image frame).

Note that the human body detection processing by the human body detection processing unit 200 is not limited to being executed via image processing. For example, the human body detection inference processing unit 201 may use a neural network and a learned model obtained via deep learning to execute inference processing to implement the human body detection processing.

In a case where a human body is detected at a plurality of sections in the frame of the optical image by image processing or human body detection processing based on inference processing using a learned model, the optical image analysis unit 105 may compare the positions of the human bodies at the plurality of sections and detect the human body captured at a position close to the central portion of the frame as the subject.

In a case where a subject (human body) is detected in the optical image, the human body detection processing unit 200 notifies the optical image analysis unit 105. At this time, the optical image analysis unit 105 executes moving body detection processing using the moving body detection processing unit 210 on the optical image in which the subject (human body) was detected. For example, the moving body detection processing unit 210 executes moving body detection processing on the human body in the optical image. In this manner, the movement (presence or absence of movement) of the detected subject can be monitored.

The moving body detection processing unit 210 executes moving body detection processing to determine whether there is a moving body in the optical image obtained from the optical image obtaining unit 103. The moving body detection processing unit 210 executes image processing using a plurality of frames of the optical image or inference processing using a learned model to determine whether the subject detected by the human body detection processing is a moving body with a movement amount exceeding the predetermined movement amount. The moving body detection processing unit 210 obtains a between-frame difference in the optical image, for example, as the moving body detection processing by image processing using a plurality of frames of the optical image and, in a case where there is a difference between frames, determines that there is an object (moving body) moving between frames of the optical image.

Also, the moving body detection processing is not limited to being executed via image processing (between-frame difference). For example, the moving body detection inference processing unit 211 may use a neural network and a learned model obtained via deep learning to execute inference processing to implement the moving body detection processing. Here, in the moving body detection via the moving body detection processing via image processing (between-frame difference) or via inference processing using a learned model, the reference (threshold) for determining whether or not there is a moving body can be changed via a setting input from the operation unit 160. For example, the reference (threshold) setting can be changed so that a change in the body position caused by a change in the condition of the subject or the like can be detected while excluding slight movements that occur after the subject is positioned for radiation imaging. In this manner, the movement of the subject desired by the operator (user) can be efficiently detected.

In a case where a moving body is detected in the optical image, the moving body detection processing unit 210 notifies the optical image analysis unit 105. The optical image analysis unit 105 notifies the determination unit 107 of the information processing apparatus 100 of the determination result of the human body detection processing unit 200 and the moving body detection processing unit 210. The determination unit 107 of the information processing apparatus 100 determines whether to display the optical image on the display unit on the basis of the notification content of the optical image analysis unit 105. Here, the determination result of each processing unit notified by the optical image analysis unit 105 may be the determination result of the human body detection processing unit 200 or the moving body detection processing unit 210 or determination results of processing other than that described above may be combined.

For example, in a case where a human body is not detected by the human body detection processing unit 200, the optical image analysis unit 105 may notify the determination unit 107 of the detection result (no detection of a human body) of the human body detection processing unit 200. Alternatively, in a case where a human body is detected by the human body detection processing unit 200 and determination of whether or not it is a moving body is performed by the moving body detection processing, the optical image analysis unit 105 may notify the determination unit 107 of the detection result (detection of a human body) of the human body detection processing unit 200 and the determination result (whether or not the detected human body is a moving body) of the moving body detection processing unit 210.

Also, as the determination results of processing other than the processing described above, a monitoring optical image from a monitoring camera 170 described below in the second embodiment and sensor data obtained from a sensor (for example, an infrared sensor, an ultrasonic sensor, a heat sensor, an acceleration sensor, or the like) are analyzed by the sensor data analysis unit 109, and the determination unit 107 may be notified of a determination result obtained by combining the analysis result of the sensor data analysis unit 109 and the analysis result of the optical image analysis unit 105. By combining the analysis result of a monitoring optical image and the analysis result of various sensor data in addition to the analysis result of the optical image, the likelihood of a false determination by the human body detection processing unit 200 and the moving body detection processing unit 210 can be reduced, and determination accuracy can be improved.

### Processing Flow

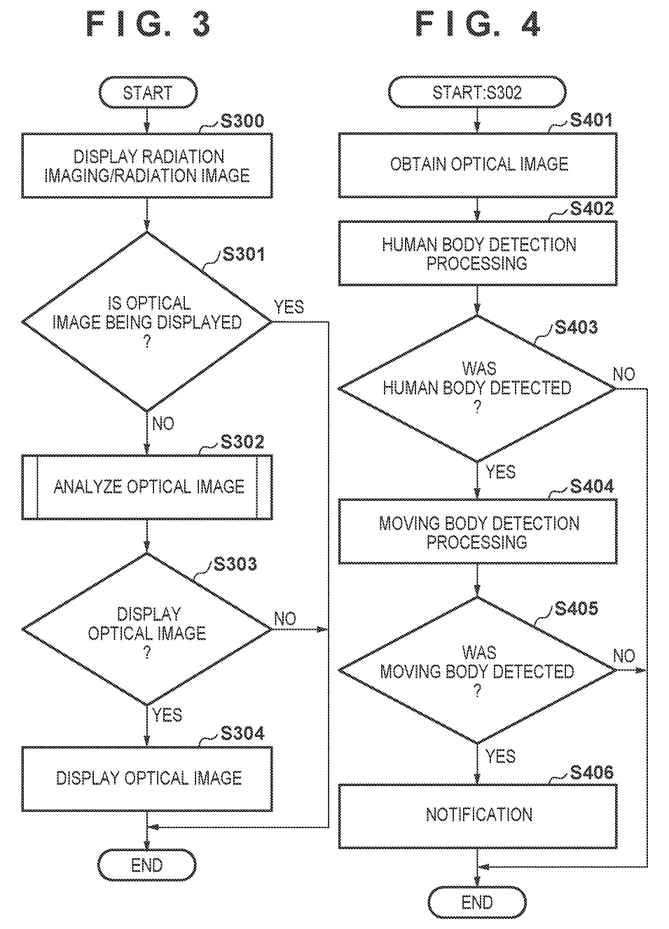

Next, the determination processing of the optical image display using the optical imaging apparatus 140 according to the present embodiment will be described in accordance with the flowchart of FIG. 3. In the example described here, for the determination processing of the optical image display illustrated in FIG. 3, the processing of S301 to S304 is described as being performed in a state in which radiation imaging is being performed and a radiation image is being displayed (S300). However, the determination processing of the optical image display according to the present embodiment is not limited to this example. For example, in a case where there is a change in the condition of the subject after positioning of the subject is complete but before the exposure switch is pressed by the operator (before the start of radiation imaging), an optical image can be displayed and a notification of the change in the condition of the subject can be swiftly performed. Alternatively, in a case where there is a change in the condition of the subject after radiation imaging is complete and when the captured radiation image is displayed on the display unit 150 and the operator is checking the radiation image, an optical image can be displayed and a notification of the change in the condition of the subject can be swiftly performed.

In S300, radiation imaging is performed and the radiation image is displayed on the display unit 150. In the present step, the image data generated by the radiation detection apparatus 130 is received by the radiation image obtaining unit 101, and image processing is executed on the image data received by the radiation image obtaining unit 101. The display control unit 104 causes the display unit 150 to display the radiation image after image processing by the image processing unit 102. Note that the radiation image obtained via radiation imaging may be a still image or moving images of fluoroscopy imaging.

In S301, the determination unit 107 of the information processing apparatus 100 determines whether to display the optical image on the display unit 150. In a case where the optical image is not displayed on the display unit 150 (No in S301), the processing advances to S302. On the other hand, in a case where the optical image is determined to be displayed on the display unit 150 in S301 (Yes in S301), the processing ends.

In S302, the optical image analysis unit 105 executes optical image analysis processing. The details of the optical image analysis processing will be described below with reference to FIG. 4.

In S303, the determination unit 107 of the information processing apparatus 100 determines whether to display the optical image on the display unit 150 on the basis of the result of the optical image analysis processing of S302 by the optical image analysis unit 105. The determination unit 107 of the information processing apparatus 100 determines whether to display the optical image on the display unit 150 on the basis of the human body detection processing and the moving body detection processing in the optical image analysis unit 105. In the moving body detection by moving body detection processing via image processing (between-frame difference) or via inference processing using a learned model executed by the optical image analysis unit 105, the degree of change in the movement of the subject can be changed via a setting input from the operation unit 160. For example, the determination unit 107 of the information processing apparatus 100 determines to display the optical image on the display unit 150 when there is a large movement exceeding the predetermined movement amount in the subject detected in a frame of the optical image.

At this time, as a reference used when determining to display the optical image, the movement amount (degree of change of movement) of the subject can be changed via a setting input from the operation unit 160. Accordingly, whether to display the optical image can be changed via a setting according to the degree of movement of the subject. For example, even in a case where, after positioning of the subject is complete, the subject moves and re-positioning is required (a case where the degree of movement is relatively small), the optical image may be set to be displayed for notification. Alternatively, the optical image may be set to be displayed for notification only in a case where there is a change in the condition such as the subject falling down (when the degree of movement is relatively large). In a case where it is determined to display the optical image (Yes in S303), the processing advances to S304. In a case where it is determined not to display the optical image in the determination processing of S303 (No in S303), the processing ends. In a case where the movement of the subject analyzed by the optical image analysis unit 105 is less than the predetermined movement amount, the determination unit 107 causes the display unit 150 to display the radiation image and determines to not display the optical image and the display control unit 104 causes the display unit 150 to display the radiation image on the basis of this determination.

In S304, the display control unit 104 performs display control to cause the display unit 150 to display the optical image obtained by the optical image obtaining unit 103. In a case where the movement of the subject analyzed by the optical image analysis unit 105 is greater than the predetermined movement amount, the determination unit 107 determines to display the optical image on the display unit 150 and the display control unit 104 causes the display unit 150 to display the optical image on the basis of this determination result. At this time, the display control unit 104 may change the method for displaying the optical image according to the result of the optical image analysis processing of S302.

In a case where the display control unit 104 causes the display unit 150 to display the radiation image obtained via radiation imaging and the optical image is displayed, the display control unit 104 does not display the radiation image and causes the display unit 150 to display the optical image.

In a case where the optical image is displayed, the display control unit 104 may cause the display unit 150 to display the radiation image and the optical image.

When an optical image is displayed, the display control unit 104 may further cause the display unit 150 to display additional information to notify of subject movement. The additional information may include one of a message for notifying of subject movement, and icon, or a combination of a message and an icon.

In a case where the movement of the subject exceeds a predetermined movement amount, the display control unit 104 may cause the display unit 150 to change the display color of the additional information and display it. In a case where the display control unit 104 displays an optical image, the determination unit 107 may output a notification sound for notifying of subject movement from the audio output unit 180.

In a case where the subject movement exceeds the predetermined movement amount and increases, the determination unit 107 may change the type of the notification sound or increase the volume of the notification sound and output it from the audio output unit 180.

According to a modified example of the display method, in a case where, in the analysis result of the optical image analysis unit 105, the movement of the subject is within a predetermined range greater than the predetermined movement amount set as the threshold, the display control unit 104 may cause the display unit 150 to display an optical image of a first size (reference size). In the analysis result, in a case where a large amount of movement of the subject detected exceeding the upper limit of the predetermined range, the display control unit 104 may cause the display unit 150 to display the optical image enlarged to a size (second size) greater than the first size (reference size). Also, together with the enlarged optical image, the additional information described above may be displayed or a notification sound may be output.

In a case where a plurality of optical images are obtained from the plurality of optical imaging apparatuses 140, the display control unit 104 may cause the display unit 150 to display the plurality of optical images obtained from the plurality of optical imaging apparatuses 140. In a case where the movement of the subject is within the predetermined range and the display unit 150 is caused to display the plurality of optical images or monitoring optical images, the display control unit 104 may cause the display unit 150 to display the optical image reduced to a size (third size) smaller than the first size (reference size).

In a case where the method for displaying the optical image is changed according to the degree of the movement of the subject and a large movement exceeding the upper limit of the predetermined range is detected, display control may be performed so that the optical image is displayed at a size enlarged from the first size to the second size in conjunction with display of the additional information described above or a warning via audio output.

### Detailed Flow of Analysis Processing of Optical Image

Next, analysis processing of the optical image obtained from the optical imaging apparatus 140 executed in S302 according to the present embodiment will be described in accordance with the flowchart of FIG. 4.

In S401, the optical image obtaining unit 103 obtains the optical image from the connected optical imaging apparatus 140.

In S402, the human body detection processing unit 200 executes human body detection processing on the optical image obtained from the optical imaging apparatus 140 in S401. The human body detection processing may be implemented using image processing. The human body detection inference processing unit 201 may use a neural network and a learned model obtained via deep learning to execute inference processing to implement the human body detection processing.

In S403, the human body detection processing unit 200 determines whether or not a human body (subject) has been detected in the optical image from the processing result of S402. In a case where a human body is detected (Yes in S403), the processing advances to S404. In a case where a human body is not detected (No in S403), the analysis processing ends.

In S404, the moving body detection processing unit 210 executes moving body detection processing on the optical image obtained from the optical imaging apparatus 140 in S401. The moving body detection processing may be implemented by image processing including obtaining a between-frame difference in the optical image and, if there is a difference between frames, setting the difference section to the object (moving body) moving between frames of the optical image. The moving body detection inference processing unit 211 may use a neural network and a learned model obtained via deep learning to execute inference processing to implement the moving body detection processing.

In S405, the moving body detection processing unit 210 determines whether the subject is moving from the processing result of S404. In a case where the moving body detection processing unit 210 determines that the subject is moving (Yes in S405), the processing advances to S406. In a case where the moving body detection processing unit 210 determines that the subject is not moving in the determination of S405 (No in S405), the processing ends.

In S406, the optical image analysis unit 105 notifies the determination unit 107 of the information processing apparatus 100 together with the processing result obtained via analysis by the optical image analysis unit 105.

According to the present embodiment, whether to display the optical image on the display unit 150 can be determined by a determination of whether a subject is captured in the optical image and a determination of whether the subject is moving on the basis of the analysis processing of the optical image of the optical imaging apparatus 140. Accordingly, even when the optical image is not displayed on the display unit 150, in a case where the subject moves a large amount exceeding the predetermined movement amount, the change in the condition of the subject can be detected and a notification can be swiftly performed.

Due to this efficient imaging flow, in a case where an emergency situation such as a change in the condition of the subject is detected when the optical image is not being displayed, the optical image can be displayed and a notification of the detected emergency situation can be performed.

### Second Embodiment

In the configuration according to the first embodiment described above, human body detection processing for detection the presence/absence of a subject and moving body detection processing for detecting the presence/absence of movement of the subject are executed using an optical image captured by the optical imaging apparatus 140 and, when the subject moves a large amount or the like, the optical image is displayed and a notification is performed of the change in the condition of the subject.

In the configuration according to the second embodiment described here, human body detection processing, position analysis of the subject, and moving body detection processing of the subject in a predetermined examination position are executed using sensor data obtained from various sensors and, when the subject moves a large amount, the optical image is displayed and a notification is performed of the change in the condition of the subject.

Hereinafter, to avoid describing matters already described in the first embodiment, only the different portions will be described. In the configuration example according to the present embodiment described here, human body detection processing, processing to determine whether the subject is in the examination position, and moving body detection processing are executed using the monitoring camera 170 correspond to an aspect of the sensor and a monitoring optical image (hereinafter also referred to as a monitoring camera image) captured by the monitoring camera 170 as sensor data.

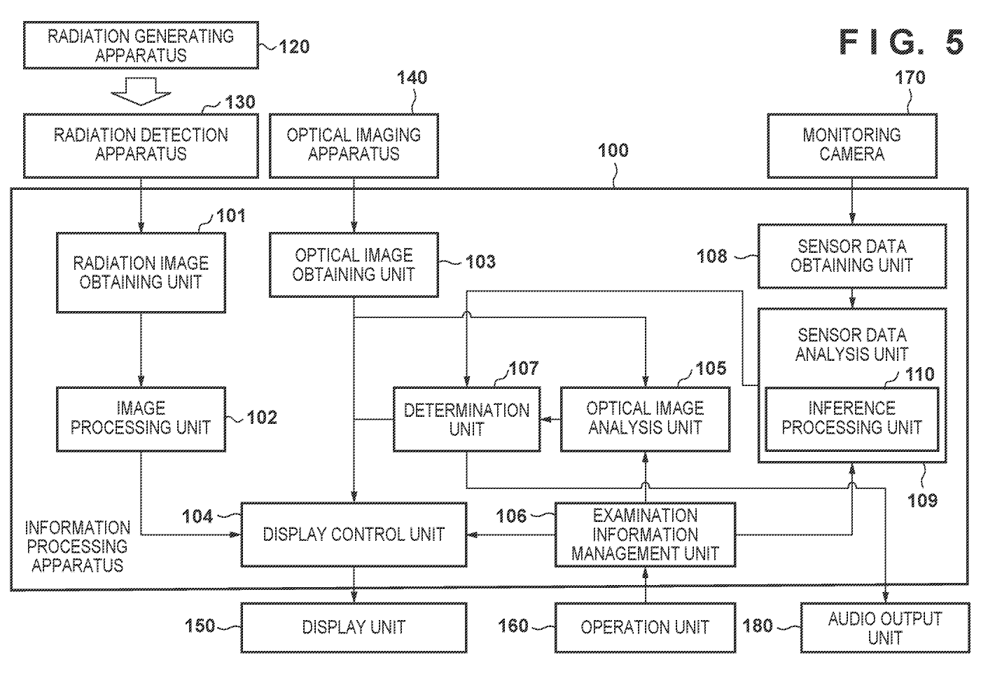

FIG. 5 is a diagram illustrating an example of the configuration of a radiation imaging system according to the present embodiment. The radiation imaging system includes the information processing apparatus 100, the radiation generating apparatus 120, the radiation detection apparatus 130, the optical imaging apparatus 140, the display unit 150, the operation unit 160, and the monitoring camera 170. The information processing apparatus 100 controls radiation imaging using the radiation detection apparatus 130 and the radiation generating apparatus 120. The information processing apparatus 100 is connected in a communication-enabling manner to the radiation detection apparatus 130, the radiation generating apparatus 120, the optical imaging apparatus 140, and the monitoring camera 170.

The monitoring camera 170 is a second optical imaging apparatus with a wider field of view (second field of view) compared to the field of view (first field of view) of the optical imaging apparatus 140 (first optical imaging apparatus). The monitoring camera 170 is disposed in an examination room where radiation imaging is performed and is able to being always capturing images of the entire examination room including radiation imaging system including the subject, the radiation generating apparatus 120, the radiation detection apparatus 130 that generates a radiation image, and the like. The monitoring camera 170 (second optical imaging apparatus) captures images of the radiation detection apparatus 130 and the subject as monitoring camera images. The monitoring camera 170 is connected to the information processing apparatus 100 via a wired or wireless network or a dedicated line. The information processing apparatus 100 controls the conditions, timing, frame rate, and the like relating to the monitoring camera image obtained by the monitoring camera 170.

A sensor data obtaining unit 108 according to the present embodiment obtains a monitoring camera image from the monitoring camera 170 as sensor data.

The sensor data analysis unit 109 analyzes the sensor data obtained from the sensor for detecting the subject. The sensor data analysis unit 109 analyzes the monitoring camera image obtained from the sensor data obtaining unit 108 and determines the absence/presence and movement of a subject in the monitoring camera image. The sensor data analysis unit 109 may determine the absence/presence and movement of the subject in the monitoring camera image via image processing executed on the monitoring camera image or inference processing using a learned model such as a machine learning or deep learning model.

The sensor data analysis unit 109 executes human body detection processing for detecting whether or not the subject is captured in the frame of the monitoring camera image obtained from the monitoring camera 170 (second optical imaging apparatus) that captures images of the radiation detection apparatus 130 that generates the radiation image and the subject as sensor data. Also, the sensor data analysis unit 109 analyzes the movement of the subject by executing determination processing to determine whether the subject detected by the human body detection processing is at the predetermined position with respect to the radiation detection apparatus 130 and moving body detection processing to determine whether the subject determined to be at the predetermined position is a moving body with movement that exceeds the predetermined movement amount.

The determination unit 107 of the information processing apparatus 100 determines whether to display the optical image obtained from the optical imaging apparatus 140 on the display unit 150 on the basis of the analysis result of the sensor data analysis unit 109. For example, the determination unit 107 of the information processing apparatus 100 analyzes the movement of the subject from the monitoring camera image obtained by the monitoring camera 170 and determines to display the optical image on the display unit 150 in a case where it is determined that the subject is a moving body with movement exceeding the predetermined movement amount (a case where it is determined that there has been a change in the condition). The display control unit 104 causes the display unit 150 to display the optical image on the basis of the determination result of the determination unit 107.

### Processing Flow

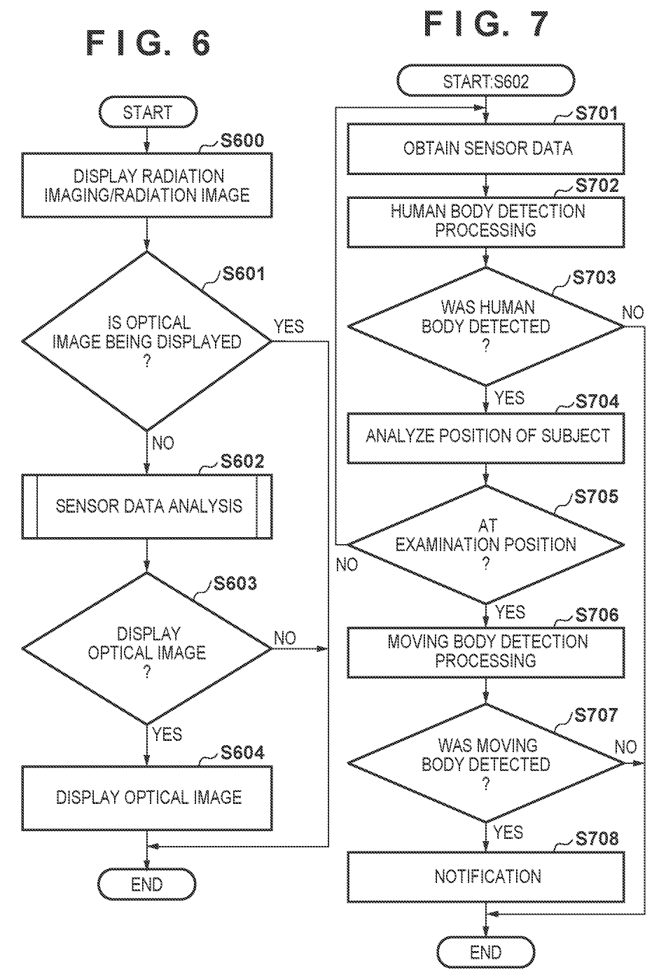

Next, the determination processing of the optical image display using the monitoring camera 170 according to the present embodiment will be described in accordance with the flowchart of FIG. 6. In the example described here, for the determination processing of the optical image display illustrated in FIG. 6, the processing of S601 to S604 is described as being performed in a state in which radiation imaging is being performed and a radiation image is being displayed (S600). However, the determination processing of the optical image display according to the present embodiment is not limited to this example. For example, in a case where there is a change in the condition of the subject after positioning of the subject is complete but before the exposure switch is pressed by the operator (before the start of radiation imaging), an optical image can be displayed and a notification of the change in the condition of the subject can be swiftly performed. Alternatively, in a case where there is a change in the condition of the subject after radiation imaging is complete and when the captured radiation image is displayed on the display unit 150 and the operator is checking the radiation image, an optical image can be displayed and a notification of the change in the condition of the subject can be swiftly performed.

In S600, radiation imaging is performed and the radiation image is displayed on the display unit 150. In the present step, the image data generated by the radiation detection apparatus 130 is received by the radiation image obtaining unit 101, and image processing is executed on the image data received by the radiation image obtaining unit 101. The display control unit 104 causes the display unit 150 to display the radiation image after image processing by the image processing unit 102. Note that the radiation image obtained via radiation imaging may be a still image or moving images of fluoroscopy imaging.

In S601, the determination unit 107 of the information processing apparatus 100 determines whether to display the optical image on the display unit 150. In a case where the optical image is not displayed on the display unit 150 (No in S601), the processing advances to S602. On the other hand, in a case where the optical image is determined to be displayed on the display unit 150 in S601 (Yes in S601), the processing ends.

In S602, the sensor data analysis unit 109 executes sensor data analysis processing. The details of the sensor data analysis processing will be described below with reference to FIG. 7.

In S603, the determination unit 107 of the information processing apparatus 100 determines whether to display the optical image on the display unit 150 on the basis of the result of the sensor data analysis processing of S602 by the sensor data analysis unit 109.

For example, the determination unit 107 of the information processing apparatus 100 determines to display the optical image on the display unit 150 when there is a change of a large movement exceeding the predetermined movement amount in the subject detected by the sensor data analysis processing. At this time, as a reference used when determining to display the optical image, the movement amount (degree of change of movement) of the subject can be changed via a setting input from the operation unit 160. In a case where it is determined to display the optical image (Yes in S603), the processing advances to S604. In a case where it is determined not to display the optical image in the determination processing of S603 (No in S603), the processing ends. In a case where the movement of the subject analyzed by the sensor data analysis unit 109 is less than the predetermined movement amount, the determination unit 107 causes the display unit 150 to display the radiation image and determines to not display the optical image and the display control unit 104 causes the display unit 150 to display the radiation image on the basis of this determination.

In S604, the display control unit 104 performs display control to cause the display unit 150 to display the optical image obtained by the optical image obtaining unit 103. In a case where the movement of the subject analyzed by the sensor data analysis unit 109 is greater than the predetermined movement amount, the determination unit 107 determines to display the optical image on the display unit 150 and the display control unit 104 causes the display unit 150 to display the optical image on the basis of this determination result. The optical imaging apparatus 140 (first optical imaging apparatus) is an optical imaging apparatus with a narrower field of view compared to the monitoring camera 170 (second optical imaging apparatus) described in the second embodiment, and the display control unit 104 causes the display unit 150 to display the optical image capturing an imaging site of the subject in radiation imaging as the optical image captured by the optical imaging apparatus 140 (first optical imaging apparatus). At this time, the display control unit 104 may change the method for displaying the optical image according to the result of the sensor data analysis processing of S602.

An example of the display control for changing the method for displaying the optical image may be similar to the processing described above in S304. For example, in a case where the method for displaying the optical image is changed according to the degree of the movement of the subject and a large movement exceeding the upper limit of the predetermined range is detected, display control may be performed so that the optical image is displayed at a size enlarged from the first size to the second size in conjunction with display of the additional information described above or a warning via audio output. In a case where the movement of the subject is within the predetermined range and the display unit 150 is caused to display the plurality of optical images or monitoring optical images, the display control unit 104 may cause the display unit 150 to display the optical image reduced to a size (third size) smaller than the first size (reference size).

Also, in a case where the display control unit 104 causes the display unit 150 to display the radiation image obtained via radiation imaging and the optical image is displayed, the display control unit 104 does not display the radiation image and causes the display unit 150 to display the optical image and the monitoring camera image (monitoring optical image).

Also, in a case where the optical image is displayed, the display control unit 104 may cause the display unit 150 to display the radiation image, the optical image, and the monitoring optical image.

### Detailed Flow of Analysis Processing of Sensor Data

Next, the analysis processing of the monitoring camera image obtained from the monitoring camera 170 executed in S602 according to the present embodiment will be described in accordance with the flowchart of FIG. 7.

In S701, the sensor data obtaining unit 108 obtains a monitoring camera image from the connected monitoring camera 170.

In S702, the sensor data analysis unit 109 executes human body detection processing on the monitoring camera image obtained from the monitoring camera 170 in S701. The human body detection processing may be implemented via image processing or may be implemented via inference processing using a neural network and a learned model obtained via deep learning.

In S703, the sensor data analysis unit 109 determines whether or not a human body (subject) has been detected in the monitoring camera image from the processing result of S702. In a case where a human body is detected (Yes in S703), the processing advances to S704. In a case where a human body is not detected (No in S703), the analysis processing ends.

In S704, the sensor data analysis unit 109 performs analysis of the position of the subject in the monitoring camera image (frame) detected in S702 using the monitoring camera image obtained from the monitoring camera 170 in S701.

In S705, the sensor data analysis unit 109 determines whether the subject is at the predetermined position for performing radiation imaging from the analysis result of S704. In the examination information managed by the examination information management unit 106, the imaging site and imaging direction for imaging the subject and the imaging environment (imaging stand, decubitus table, cassette imaging, or the like) are set. The sensor data analysis unit 109 determines whether the position of the subject in the monitoring camera image (frame) detected in S702 is the predetermined position with respect to the position of the radiation detection apparatus 130 installed in the imaging environment.

In a case where the sensor data analysis unit 109 determines that the subject is at the predetermined position for performing radiation imaging (Yes in S705), the processing advances to S706. In a case where the sensor data analysis unit 109 determines that the subject is not at the predetermined position for performing radiation imaging (No in S705), the processing returns to S701 and similar processing is repeated.

In S706, the sensor data analysis unit 109 executes moving body detection processing on the monitoring camera image obtained from the monitoring camera 170 in S701. The moving body detection processing may be implemented by image processing including obtaining a between-frame difference in the monitoring camera image and, if there is a difference between frames, setting the difference section to the object (moving body) moving between frames of the optical image. A neural network and a learned model obtained via deep learning to execute inference processing may be used to implement the moving body detection processing.

In S707, the sensor data analysis unit 109 determines whether the subject is moving from the processing result of S706. In a case where the sensor data analysis unit 109 determines that the subject is moving (Yes in S707), the processing advances to S708. In a case where the sensor data analysis unit 109 determines that the subject is not moving in the determination of S707 (No in S707), the processing ends.

In S708, the sensor data analysis unit 109 notifies the determination unit 107 of the information processing apparatus 100 together with the processing result obtained via analysis by the sensor data analysis unit 109.

According to the present embodiment, it can be determined whether to display on the display unit 150 an optical image captured by the optical imaging apparatus 140 from the absence/presence, position, and movement of the subject obtained from image analysis processing executed on the monitoring camera image of the monitoring camera 170 capturing images of the entire examination room. Accordingly, even if the optical image is not being displayed on the display unit 150 via analysis processing of the optical image analysis unit 105, in a case where the subject at a predetermined position for performing radiation imaging moves a large amount exceeding the predetermined movement amount, a change in the condition of the subject is detected, or the like via analysis processing of the sensor data analysis unit 109, the optical image captured by the optical image obtaining unit 103 from the optical imaging apparatus 140 can be caused to be displayed on the display unit 150. In this manner, when the subject moves a large amount exceeding the predetermined movement amount or when a change in the condition of the subject is detected, a notification can be swiftly performed.

Note that in the configuration according to the present embodiment described above, human body detection processing and moving body detection processing are executed using a monitoring camera image obtained from the monitoring camera 170 as an aspect of sensor data obtained from a sensor. However, the configuration is not limited to the monitoring camera 170 and various sensors can be used as long as a subject can be detected and the absence/presence of movement of the subject can be detected. The sensor includes a first sensor that detects the subject as a human body and detects whether the subject is at the predetermined position with respect to the radiation detection apparatus 130 and a second sensor that detects whether the subject detected at the predetermined position is a moving body exceeding the predetermined movement amount. The first sensor may include, for example, an infrared sensor, an ultrasonic sensor, or a temperature sensor. The second sensor may include, for example, an acceleration sensor. The first sensor (infrared sensor, ultrasonic sensor, temperature sensor, or the like), for example, is disposed at or near the radiation generating apparatus 120 so that the radiation detection apparatus 130 and the subject are within the detection range of the sensor. The second sensor (acceleration sensor or the like), for example, is worn by the subject so that movement of the subject can be detected. The sensor data obtaining unit 108 can obtain the sensor data of the first sensor via wired or wireless communication and can obtain the sensor data of the second sensor via wireless communication.

For example, as processing corresponding to the processing of S701 to S705 of FIG. 7, infrared rays emitted from the subject may be detected and obtained as sensor data using an infrared sensor or the like. The sensor data analysis unit 109 may obtain the distance (distance information) between the radiation detection apparatus 130 and the subject and may analyze whether the subject is at the predetermined position for performing radiation imaging on the basis of the obtained infrared sensor data. Alternatively, heat emitted from the subject may be detected and obtained as sensor data using a heat sensor or the like. The sensor data analysis unit 109 may obtain the distance (distance information) between the radiation detection apparatus 130 and the subject and may analyze whether the subject is at the predetermined position for performing radiation imaging on the basis of the obtained heat sensor data. Alternatively, the distance from the subject may be measured using an ultrasonic sensor or the like and reflected ultrasonic waves. The sensor data analysis unit 109 may obtain the distance (distance information) between the radiation detection apparatus 130 and the subject and may analyze whether the subject is at the predetermined position for performing radiation imaging using reflected ultrasonic waves. Also, the sensor used for detecting a human body may be implemented via a single sensor or a combination of a plurality of types of sensors.

For example, as the processing corresponding to the processing of S706 to S708 of FIG. 7, moving body detection processing using an acceleration sensor may be executed. The sensor data analysis unit 109 may execute moving body detection processing on the basis of the detection result from the acceleration sensor. The sensor data analysis unit 109 determines that the subject is a moving body in a case where the movement of the subject is determined to exceed a threshold via moving body detection processing based on the acceleration data from the acceleration sensor. Human body detection processing and moving body detection processing may be implemented via combined processing using a combination of sensors for processing for human body detection processing and moving body detection processing.

According to the present embodiment, due to this efficient imaging flow, in a case where an emergency situation such as a change in the condition of the subject is detected when the optical image is not being displayed, the optical image can be displayed and a notification of the detected emergency situation can be performed.

According to the present disclosure, in a case where a change in the condition of the subject is detected such as when movement exceeds the predetermined movement amount, the optical image can be displayed and a notification of the change in the condition of the subject can be performed.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2024- 180340, filed October 15, 2024 which is hereby incorporated by reference herein in its entirety.

What is claimed is:

1. An information processing apparatus that processes a radiation image obtained via radiation imaging of a subject, comprising:
an analysis unit configured to perform an analysis of movement of the subject in an optical image obtained from a first optical imaging apparatus;
a determination unit configured to perform a determination of whether to display the optical image on a display unit, based on the analysis; and
a display control unit configured to perform control display of the display unit, based on the determination.

2. The information processing apparatus according to claim 1, wherein
the analysis unit analyzes movement of the subject by executing
human body detection processing for detecting the subject captured in a frame of the optical image by executing image processing or inference processing using a learned model, and
moving body detection processing for determining whether the subject detected by the human body detection processing is a moving body exceeding a predetermined movement amount by executing image processing using a plurality of frames of the optical image or inference processing using a learned model.

3. The information processing apparatus according to claim 2, wherein
in a case where a human body is detected at a plurality of sections in the frame via the human body detection processing, the analysis unit compares positions of human bodies at the plurality of sections and detects a human body imaged at a position close to a center of the frame as the subject.

4. The information processing apparatus according to claim 2, wherein
in a case where a plurality of human bodies different from one another are detected in the frame via the human body detection processing, the analysis unit obtains feature information of the plurality of human bodies and detects one of the plurality of human bodies as the subject, based on the feature information.

5. The information processing apparatus according to claim 2, wherein
the predetermined movement amount is a threshold changeable via a setting, and
in a case where movement of the subject detected via the moving body detection processing exceeds the threshold, the analysis unit determines the subject to be a moving body.

6. The information processing apparatus according to claim 2, wherein
in a case where movement of the subject analyzed by the analysis unit is less than a predetermined movement amount, the determination unit determines, via a determination, to cause the display unit to display the radiation image and not display the optical image, and
the display control unit causes the display unit to display the radiation image, based on the determination.

7. The information processing apparatus according to claim 2, wherein
in a case where movement of the subject analyzed by the analysis unit is greater than a predetermined movement amount, the determination unit determines, via a determination, to cause the display unit to display the optical image, and
the display control unit causes the display unit to display the optical image, based on the determination.

8. The information processing apparatus according to claim 7, wherein
the display control unit
causes the display unit to display the radiation image obtained by the radiation imaging, and
in a case of displaying the optical image, causes the display unit to not display the radiation image and to display the optical image.

9. The information processing apparatus according to claim 7, wherein
in a case of displaying the optical image, the display control unit causes the display unit to display the radiation image and the optical image.

10. The information processing apparatus according to claim 8, wherein
in a case of displaying the optical image, the display control unit further causes the display unit to display additional information notifying of movement of the subject.

11. The information processing apparatus according to claim 10, wherein
the additional information includes one of a message for notifying of movement of the subject, an icon, or a combination of the message and the icon.

12. The information processing apparatus according to claim 10, wherein
in a case where movement of the subject is a large amount exceeding a predetermined movement amount, the display control unit changes a display color of the additional information and causes the display unit to display the additional information.

13. The information processing apparatus according to claim 7, wherein
in a case of the display control unit displaying the optical image, the determination unit causes a notification sound for notifying of movement of the subject to be output from an audio output unit.

14. The information processing apparatus according to claim 13, wherein
in a case where movement of the subject is a large amount exceeding a predetermined movement amount, the determination unit changes a type of the notification sound or increases a volume of the notification sound and causes the notification sound to be output from the audio output unit.

15. The information processing apparatus according to claim 7, wherein
in an analysis result of the analysis unit, in a case where movement of the subject is within a predetermined range exceeding the predetermined movement amount set as a threshold, the display control unit causes the display unit to display an optical image of a first size, and
in the analysis result, in a case where a large amount of movement of the subject is detected and the large amount of movement exceeds an upper limit of the predetermined range, the display control unit causes the display unit to display an optical image enlarged to a second size larger than the first size.

16. The information processing apparatus according to claim 7, wherein
in a case where a plurality of optical images obtained from a plurality of the first optical imaging apparatuses are obtained, the display control unit causes the display unit to display the obtained plurality of optical images.

17. The information processing apparatus according to claim 1, further comprising a data analysis unit configured to analyze sensor data obtained from a sensor configured to detect the subject, wherein
the data analysis unit analyzes movement of the subject by executing
human body detection processing for detecting whether the subject is captured in a frame of a monitoring optical image obtained from a second optical imaging apparatus configured to image a radiation detection apparatus configured to generate the radiation image and the subject as the sensor data,
determination processing for determining whether the subject detected by the human body detection processing is at a predetermined position with respect to the radiation detection apparatus, and
moving body detection processing for determining whether the subject determined to be at the predetermined position is a moving body exceeding a predetermined movement amount.

18. A radiation imaging system comprising:
a radiation detection apparatus; and
the information processing apparatus according to claim 1 connected in a communication-enabling manner to the radiation detection apparatus.

19. An information processing method for an information processing apparatus that processes a radiation image obtained via radiation imaging of a subject, comprising:
performing an analysis of movement of the subject in an optical image obtained from a first optical imaging apparatus;
performing a determination of whether to display the optical image on a display unit, based on the analysis; and
performing control display of the display unit, based on the determination.

20. A non-transitory computer-readable storage medium storing a program for causing a computer to execute the information processing method according to claim 19.
